(19) United States
(12) Patent Application Publication
(10) Pub. No.: US 2023/0329981 A1
(43) Pub. Date: Oct. 19, 2023

(54) SOLID HYDRATING COMPOSITION

(71) Applicant: L V M H RECHERCHE, Saint Jean De Braye (FR)

(72) Inventors: Murielle Farigoule, Saint Jean De Braye (FR); Valérie Bouchard De La Poterie, Saint Jean De Braye (FR)

(21) Appl. No.: 18/027,163

(22) PCT Filed: Sep. 21, 2021
(86) PCT No.: PCT/FR2021/051615

§ 371 Date: Mar. 20, 2023

(30) Foreign Application Priority Data
Sep. 21, 2020 (FR) FR2009560

(51) Int. Cl. A61K 8/02; A61Q 1/10; A61Q 1/08

(52) U.S. Cl. CPC A61K 8/022; A61Q 1/08; A61Q 1/10; A61K 2800/43

(57) ABSTRACT

The present invention concerns a cosmetic composition for caring for and/or making up keratinous substances, more particularly the skin, in the form of a solid composition comprising, in a physiologically acceptable medium, at least: water in a an amount of from 5% to 50% by weight of water, at least one hydroxyl compound selected from polyols, glycol ethers and mixtures thereof, a particulate solid phase comprising at least fillers, and optionally an oily phase comprising at least one oil, said composition being obtainable by a method comprising the following steps: (i) mixing the particulate phase, the hydroxyl compound and the water, and the optional oily phase constituting the liquid phase, to form a paste; and (ii) shaping the paste by compacting and partial removal of the liquid phase, to give the composition in the form of a solid composition.

### FIELD OF THE INVENTION

The present invention concerns the cosmetics field and in particular to the field of hydrating and fresh solid compositions, particularly intended for cosmetic care and/or for making up the skin and/or the lips.

### STATE OF THE ART

Known from the state of the art are hydrating compositions in liquid form, such as lotions, gels, oil-in-water emulsions, comprising a high rate of aqueous phase and in particular a high rate of water, for imparting a fresh and hydrating effect to the composition.

However, to the Applicant’s knowledge, there is no equivalent in the field of solid compositions, α fortiori when they contain a pulverulent phase such as filters and/or pigments. Cosmetic compositions based on powders, whether in free or compact form, are often characterized by a dry sensation when applied to keratin materials. Consumers are looking for more comfort when applied, in particular those with dry and/or fragile skin, with a beneficial effect on the skin (care, make-up) without a feeling of discomfort or dryness when applied.

Therefore, there remains the need to develop new solid compositions comprising a pulverulent phase (for example: filters and/or pigments) intended to provide softness, coverage and/or color to keratin materials, but which are hydrating and fresh.

The Applicant has precisely developed hydrating and fresh solid compositions comprising rates of aqueous phase which are unequaled in compositions based on pulverulent materials and volatile or non-volatile binders. The solid hydrating composition of the invention, obtained by a method of the “slurry” type, in fact comprises an aqueous phase in a total amount which can range up to 60% by weight relative to the total weight of the composition.

According to a particular embodiment, the composition is obtained by wet method, that is to say that the pulverulent materials and the binder are diluted in a solvent to obtain a paste (otherwise called slurry) which is then shaped and dried. ‘Slurry’ is a term used to designate a cosmetic paste (suspension) composed of a particulate phase (fillers and pigments), a volatile or non-volatile liquid phase to provide smoothness to the powder and a solvent, which will be sucked and/or partially evaporated during the method. The combination of these elements allows to obtain unprecedented comfort and freshness at the time of application of the composition to the skin, and produces both a visually attractive makeup effect and a skincare effect, particularly by improving their state of hydration.

‘Composition according to the invention’ will generally mean the final cosmetic composition obtained according to the method of the invention, to be distinguished from the paste or slurry, which is obtained in an intermediate step of the method.

### DISCLOSURE OF THE INVENTION

An object of the invention is to propose new compositions comprising a solid phase consisting of powders, water, and at least one hydroxyl compound selected from polyols, glycol ethers and mixtures thereof, which has a hydrating effect on keratin materials. The composition may further comprise, optionally, an oily phase to improve the texture.

‘Solid composition’ according to the invention means in particular a composition having a hardness of from 100 to 500 Gf (gram force), in particular from 200 to 400 Gf (gram force). The hardness of a composition can be measured according to the following protocol.

The hardness is measured using a TA.XT PLUS TEXTURE ANALYSER (STABLE MICROSYSTEMS) texturometer, equipped with a rod on which is fixed a cylinder with a diameter of 5 mm which is applied to the surface of the compact. The cylinder penetration force is measured over a thickness of 1 mm at a speed of 0.5 mm/second.

‘Solid hydrating composition’ according to the invention means in particular a composition conferring a positive variation in hydration of the skin to which the composition is applied, in particular a variation of from +1% to +40%, in particular +5% to +30%, or even +10% to +25%. This variation in hydration can in particular be evaluated by the following protocol.

The hydration is measured by corneometry, using a CORNEOMETER CM825 apparatus supplied by the company Courage & Khazaka SN “NUM_SERIE”, with which the person skilled in the art is familiar. The method for measuring the hydration of the stratum corneum is based on the creation of an electric field on the surface of the skin and the detection, using electrodes, of variations in the dielectric constant induced by the state of hydration of the upper layer of the epidermis (see Measurement of the electrical properties is recognized as an objective method of assessing stratum corneum hydration (E. Berardesca - EEMCO guidance for the assessment of stratum corneum hydration: electrical methods - Skin Research and Technology 1997; 3:126-132). The composition is applied to the forearm of a volunteer. The quantity applied corresponds to 2 µl/cm2 of skin. The area of application is then protected from rubbing using a plastic disk 5 cm in diameter and 1 cm in height fixed with a plaster. A measurement is carried out on the application area, before application of the composition, then 6 hours after said application of the composition, leaving a period of 15 minutes for stabilization after removal of the anti-friction protection, before the measurement. The residual film of the composition is wiped from the application area. The variation in hydration is calculated from the measurements carried out according to the method described previously.

‘Fresh composition’ means according to the invention a composition producing, at the time of its application to the skin, a feeling of freshness, felt by the user as pleasant and providing well-being. This sensation of freshness is produced in particular by the evaporation of the water in the composition at the time of its application.

The solid composition of the invention advantageously has a creamy and hydrating nature when taken and when applied to keratin materials, unlike conventional compact powders characterized by a powdery and dry appearance.

For this purpose, according to a first aspect of the invention, a cosmetic composition for caring for and/or making up keratin materials, more particularly the skin, is proposed in the form of a solid hydrating composition comprising, in a physiologically acceptable medium, at least:
water in an amount of from 5% to 50% by weight relative to the total weight of the composition,
at least one hydroxyl compound selected from polyols, glycol ethers and mixtures thereof,
a particulate solid phase comprising at least fillers, and
optionally an oily phase comprising at least one oil, said composition being obtainable by a method comprising the following steps: (i) mixing the particulate phase, and the hydroxyl compound, the water and the optional oily phase constituting the liquid phase, to form a paste; and (ii) shaping the paste by compacting and partial removal of the liquid phase to give the composition in the form of a solid composition.

The shaping the paste may or may not be carried out in a container.

Preferably, said composition is obtained by a method comprising the following steps: (i) mixing the particulate phase, and the hydroxyl compound, the water and the optional oily phase constituting the liquid phase, to form a paste; and (ii) shaping the paste by compacting and partial removal of the liquid phase (consisting of volatile and/or non-volatile liquids) to give the composition in the form of a solid composition.

The invention also relates a cosmetic method for caring for and/or making up keratin materials, more particularly the skin and/or the lips, comprising the application to said keratin materials of a composition as defined according to the invention.

“Keratin materials” according to the invention means the skin and/or its appendages, and the lips. In particular, it will be the skin of the face, the neck and/or the body, and the lips. In particular, healthy keratin materials means not having disorders or conditions that would come under a pathological state (‘non-healthy’ subjects, suffering from a pathology).

### DETAILED DESCRIPTION OF THE INVENTION

The invention therefore relates, according to a first aspect, to a cosmetic composition for caring for and/or making up keratin materials, more particularly the skin, in the form of a solid composition comprising, in a physiologically acceptable medium, at least:
water in an amount of from 5% to 50% by weight of water relative to the total weight of the composition,
at least one hydroxyl compound selected from polyols, glycol ethers and mixtures thereof,
a particulate solid phase comprising at least fillers, and
optionally an oily phase comprising at least one oil, said composition being obtainable by a method comprising the following steps: (i) mixing the particulate phase, and the hydroxyl compound, the water and the optional oily phase constituting the liquid phase, to form a paste; and (ii) shaping the paste by compacting and partial removal of the liquid phase to give the composition in the form of a solid composition.

The shaping of the paste may or may not be carried out in a container, such as a silicone mold or a bucket.

According to a particular and preferred embodiment, the composition according to the invention is obtained by a method comprising the following steps: (i) mixing the particulate phase, and the hydroxyl compound, the water and the optional oily phase constituting the liquid phase, to form a paste; and (ii) shaping the paste by compacting and partial removal of the liquid phase to give the composition in the form of a solid composition.

‘Partial removal of the liquid phase (present in the paste)’ according to the invention, means that the solid hydrating composition obtained comprises a final rate of water sufficient to give it the desired hydrating properties. The liquid phase consists of water, the hydroxyl compound and any additional volatile and non-volatile solvents and binders (oils). Thus, the liquid phase according to the invention can consist of volatile liquids and/or non-volatile liquids at room temperature. The remainder of the description will refer to the removal of the liquid phase or the removal of the volatile or non-volatile liquid.

This removal step is controlled so as to obtain the desired final percentage of water in the composition. Reference will thus be made to ‘controlled removal’ or ‘controlled suction’ in the illustrative examples of the invention.

This step of partially removing the liquid phase allows to remove the liquid phase in a controlled manner in order to solidify the composition. The partial removal of the liquid phase can be carried out with mechanical means such as suction, or thermal means such as heat.

The partial removal of the liquid phase can also comprise the combination of the means described previously. Thus, in accordance with a variant of the method of the invention, the partial removal of the liquid phase contained in the paste comprises a mechanical removal followed by a thermal removal.

The method may for example comprise two successive steps: partially drying the paste using mechanical removal means to obtain a wet powder, and drying the wet powder by thermal means. The mechanical removal can be carried out by suction under a press, and the thermal removal can be carried out for example by drying in an oven, preferably at a temperature below 70° C., more preferably still below 60° C.

Thus the solid hydrating composition will comprise an amount of 5% to 50% of water relative to the total weight of said composition.

According to a particular embodiment, the partial removal of the liquid phase is done by suction under vacuum, at room temperature and in particular with a compression pressure between 1 and 4 bars and a suction pressure between 1 and 4 bars (limits included).

The person skilled in the art will be able to adapt the duration of this step to optimize the partial removal of the liquid phase. According to a particular embodiment, the partial removal of the liquid phase is done by suction under vacuum, at ambient temperature at a compression pressure of 1 to 2 bars and a suction pressure of 1 to 2 bars, for 3 to 20 seconds, in particular a compression pressure of 2 bar and a suction pressure of 1 bar for 20 seconds.

The paste can comprise a higher or lower proportion of liquid depending on the duration and the means of removal used.

The water thus preferably represents from 10% to 70% by weight of the paste, and from 5% to 50% by weight of the final solid composition according to the invention.

Thus, the water amount of the solid compositions of the invention will generally range from 5% to 50% by weight of water, in particular from 10% to 45% by weight of water relative to the total weight of said composition.

“Physiologically acceptable medium” means a medium compatible with keratin materials, particularly the skin and/or the lips.

### Liquid Phase

The liquid phase according to the invention may consist of volatile liquids and/or liquids which are not volatile at ambient temperature.

### Water

The solid hydrating composition of the invention comprises at least water.

The water amount of the solid hydrating compositions of the invention will generally range from 5% to 50% by weight of water relative to the total weight of said composition. According to a particular and preferred embodiment, the water amount in the composition of the invention ranges from 10% to 50%, in particular from 20% to 45%, or even from 30% to 40% by weight relative to the total weight of the composition.

### Hydroxyl Compound

### Polyols and Glycol Ethers

The solid hydrating composition of the invention comprises a liquid phase comprising at least one hydroxyl compound selected from a polyol, a glycol ether and mixtures thereof. Mention may in particular be made of:
C3-C6 polyols, for example a C3-C6 aliphatic diol (or glycol) such as propane-1,2-diol (or propylene glycol), propane-1,3-diol, a butylene glycol, pentylene glycol (pentane-1,5-diol), or a triol such as glycerol,
a glycol ether or an aromatic alcohol such as phenoxyethanol, and mixtures thereof.

Preferably, the composition comprises at least one C3-C5 polyol, preferably glycerol.

The total amount of polyol(s) and/or glycol ether(s) in the solid hydrating composition of the invention will in particular range from 1% to 30% by weight relative to the total weight of the composition, in particular from 1% to 25% by weight relative to the total weight of the composition. According to a particular embodiment, the total amount of polyol(s) and/or glycol ether(s), in particular of glycerol, ranges from 5% to 20% by weight, preferably from 8% to 15% by weight relative to the total weight of the composition.

According to a particular and preferred embodiment, the water will be present in the composition in an amount of from 5% to 50% by weight, preferably between 20% and 40% by weight relative to the total weight of the composition and the glycerol will be present in the composition in an amount of from 2% to 45% by weight, preferably between 5% and 15% by weight relative to the total weight of the composition.

### Oil Phase (Optional)

The liquid phase of the composition according to the invention may further optionally comprise, an oily phase comprising at least one oil.

“Oily phase” means an oil or a mixture of oils which are miscible with each other.

“Oil” means, within the meaning of the invention, a fatty substance, insoluble in water, liquid at 25° C. and atmospheric pressure.

These oils can be volatile or non-volatile, vegetable, mineral or synthetic oils.

The oily phase can comprise binders of various kinds such as fatty esters, silicones, hydrocarbon oils, butters, and mixtures thereof. According to a particular embodiment, use will be made of fatty esters, hydrocarbon oils, and mixtures thereof.

These ingredients play the role of binders of the particulate solid phase at the time of compaction of the paste and provide cohesion, and in terms of texture, creaminess, comfort when applied and a care side, non-drying to the powder.

As hydrocarbon oils, mention may in particular be made of linear or branched C₈ -C₁₆ alkanes, branched C₈ -C₁₆ esters, hydrocarbon-based oils of plant origin, C₁₀-C₄₀ synthetic ethers, C₁₀-C₄₀ synthetic esters, C₁₂-C₂₆ fatty alcohols, C₁₂-C₂₂ higher fatty acids, and mixtures thereof.

Mention may in particular be made of isononyl isonanoate, pentaerythrityl triisostearate, isohexyl neopentanoate or a hydrocarbon such as isododecane, isodecane, isohexadecane, n-dodecane (C₁₂) and n-tetradecane (C₁₄) or the undecane-tridecane mixture.

As silicone oils, mention may in particular be made of linear or cyclic silicone oils, phenyl or non-phenyl silicone oils, and mixtures thereof.

### Structuring Agents

The composition of the invention may optionally comprise agents for structuring or gelling the liquid phase, in particular hydrophilic gelling agents (or suspending agents), pasty fatty substances or waxes, of animal, vegetable, mineral or synthetic origin.

By way of “hydrophilic gelling agents”, mention may in particular be made of acrylic acid polymers, polysaccharide gelling agents such as alginates, xanthan gums, carrageenan gums, agar gums, guar gums, gellan gums, sclerotium gums, chitosans, mannans, cellulose derivatives, gelatin, pectins, mineral gelling agents such as bentones or modified silicas, and mixtures thereof.

According to a particular embodiment, the solid hydrating composition also comprises at least one hydrophilic gelling agent selected from a bentonite gel, a hectorite gel, and mixtures thereof.

### Film-Forming Agents

The composition can advantageously further comprise a film-forming agent, preferably a film-forming polymer.

Film-forming polymer means a polymer capable of forming a continuous film on a support. In the text, the word polymer can denote a homopolymer or a copolymer. “Copolymer” means a polymer comprising at least two monomers or two different blocks, which may be of the same chemical family but of different structure.

The film-forming agents have an ability to form a film on the surface of the skin at the time of application of the composition, and allow to obtain an improvement in the staying power over time and in the resistance to sebum, to perspiration or mechanical stresses, in particular related to facial movements. The formation of this film thus allows to improve and extend the staying power of the composition, and therefore the effect produced by said composition, but also to give it a property called “non-transfer” property.

The compositions according to the invention comprising at least one such film-forming agent are referred to as “long-lasting” and/or “non-transferring”.

These film-forming agents can be dissolved in a liquid phase according to their affinity or else be dispersed in the form of particles or latex in a phase in which they are insoluble. These film-forming agents are advantageously polymers which can be hydrophilic in nature and thus have an affinity for the aqueous phase, or hydrophobic in nature and have an affinity for the optional oily phase of the composition.

The hydrophilic character is in particular conferred by the presence of oxygen or nitrogen atoms in the molecule, in the form of hydroxyl, carboxylic, acrylic, ester, amide, urethane or other groups comprising heteroatoms or else an ether or ketone function.

The hydrophobic character is related to the absence of oxygen or nitrogen atoms and more generally of heteroatoms in the molecule, and the exclusive presence of saturated or unsaturated, cyclic, linear or branched hydrocarbon groups.

Hydrophilic film-forming agents can advantageously be selected from synthetic compounds such as polyurethanes and their derivatives, and more particularly aqueous polyurethane dispersions which may have a solid amount (dry matter) of 20 to 60% by weight, in particular polyurethane-35, polyalkylenes, such as polyisoprene.

Such polyurethane dispersions are for example Polyurethane-34 sold under the commercial references Baycusan® C1000, C1001 by the company BAYER, and Polyurethane-35, which comprises 41% of polyurethane in dry matter in dispersion in water, sold under the commercial reference Baycusan® C1004 by the company BAYER (COVESTRO).

It is also advantageously possible to choose hydrophilic film-forming agents from compounds that are natural or of natural origin, such as native or chemically modified polysaccharides, and in particular pullulan or one of its derivatives, a starch or one of its derivatives and mixtures thereof.

According to a particular embodiment, the composition comprises a hydrophilic film-forming polymer, advantageously selected from native or chemically modified polysaccharides.

According to a particularly preferred variant, the film-forming polymer is selected from pullulan or its derivatives, a starch or its derivatives, in particular hydroxypropyl starch.

### A Particularly Preferred Starch Derivative Is Hydroxypropyl Starch (INCI Hydroxypropyl Starch)

According to a particularly preferred variant, the composition comprises at least one polyol or one polyol ether and at least one hydrophilic film-forming agent.

The hydrophilic film-forming polymer may be present in an amount of from 0.1 to 10% by weight, preferably from 0.5 to 5% by weight of dry matter relative to the total weight of said composition. The percentage of hydrophobic film-forming polymer is expressed as % by weight of dry extract (dry matter or active matter, a.m) relative to the total weight of the composition.

Furthermore, the composition may comprise at least one hydrophobic film-forming polymer, to improve the staying power properties of the film of the composition on the skin.

Hydrophobic or fat-soluble film-forming polymer means a film-forming polymer dissolved in the optional oily phase of the composition.

The hydrophobic film-forming polymer can be of natural or synthetic origin, and is advantageously selected from the group consisting of:
trimethylsiloxysilicates such as Belsil TMS 803 (WACKER), KF7312 from Shin Etsu
phenylalkylsiloxysilicates in which the alkyl group preferably comprises from 1 to 6 carbon atoms, such as phenylpropyldimethylsiloxysilicate,
silicone acrylate polymers such as acrylate/dimethicone copolymers, and in particular acrylate/dimethicone copolymers in cyclopentasiloxane (such as, for example, KP-545 from Shin-Etsu), acrylate/dimethicone copolymers in methyl trimethicone (such as, for example, KP-549 and KP-579 from Shin-Etsu), and acrylate/dimethicone copolymers in isododecane (such as, for example, KP-550 from Shin-Etsu); acrylate/polytrimethylsiloxy-methacrylate copolymers, and in particular acrylate/polytrimethylsiloxy-methacrylate copolymers in dimethicone (such as, for example, FA-4003 DM from Dow Corning®), acrylate/polytrimethylsiloxy-methacrylate copolymers in isododecane (such as for example the FA-4004 ID from Dow Corning®),
polyalkylsilsesquioxanes comprising from 1 to 6 carbon atoms, and preferably polymethylsilsesquioxane (such as for example Silform® Flexible Resin from Momentive),
trialkylsiloxysilylcarbamoyl pullulans in which the alkyl group comprises 1 to 6 carbon atoms, and preferably trimethylsiloxysilylcarbamoyl pullulan (such as TSPL-30-ID from Shin-Etsu),
copolymers of vinylpyrrolidone (VP) and alkene comprising from 2 to 20 carbon atoms, such as copolymers of VP/eicosene, VP/hexadecene, VP/styrene,
copolymers of a vinyl ester, and preferably copolymers of vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/octadecene-1, vinyl acetate/dodecene-1, vinyl stearate/ethyl vinyl ether, vinyl propionate/cetyl vinyl ether, vinyl stearate/allyl acetate, dimethyl-2,2-vinyl octanoate/vinyl laurate, dimethyl-2,2-allyl pentanoate/vinyl laurate, dimethyl vinyl propionate/vinyl stearate, allyl dimethyl propionate/vinyl stearate,
hydrogenated or non-hydrogenated polyolefins, and preferably polymers or copolymers of alkenes comprising from 2 to 20 carbon atoms, such as polybutenes, polyisobutenes, polydecenes,
alkylcelluloses, and preferably alkylcelluloses carrying an alkyl group comprising from 2 to 6 carbon atoms, such as ethylcellulose and propylcellulose,
polyvinyl alcohols, and
mixtures thereof.

The hydrophobic film-forming polymer may be present in an amount of from 0.1 to 10% by weight, preferably from 0.5 to 5% by weight of dry matter relative to the total weight of said composition. The percentage of hydrophobic film-forming polymer is expressed as % by weight of dry extract (dry matter or active matter, a.m) relative to the total weight of the composition.

### Additional Ingredients

The composition may further comprise at least one additional ingredient selected from dyes, surfactants, perfumes, electrolytes, antioxidants, preservatives, and physical and/or chemical UV filters.

The composition of the invention can advantageously incorporate any type of cosmetic active agent, whether hydrophilic or lipophilic. The preferred active agents are selected from the group consisting of active agents having a hydrating activity such as hyaluranic acid or ceramides, active agents having an anti-aging activity; active agents having a smoothing activity and on the skin radiance; the active agents having a skin depigmenting activity or lightening activity; active agents having a slimming activity; active agents having a hydrating activity; active agents having a soothing or relaxing activity; and mixtures thereof.

The composition is advantageously devoid of surfactant, because it is not necessary, unlike the powders of the prior art obtained by pasting powdery materials with water, to use a surfactant to guarantee a good homogeneity of the paste. Thus, according to a particular embodiment, the surfactant amount in the composition of the invention will be less than 5% by weight, in particular less than 3% by weight, preferably less than 1% by weight relative to the total weight of the composition.

According to a particular embodiment, the composition of the invention does not comprise a surfactant.

### Particulate Solid Phase

The particulate phase consists of a mixture of particles of different shapes: rods, platelets and sphericals. According to a particular embodiment, the filters in the form of platelets are in high proportion in the cosmetic formulas of the invention. To these white fillers can be added texture and touch agents such as spherical filters to provide glide. The aim is to optimize the ratio of the different powders so that they compact while maintaining satisfactory sensoriality: gliding, pick-up and softness properties.

The particulate solid phase advantageously consists of a mixture of pulverulent materials comprising at least one pigment and/or at least one filler. The pigments can be selected from mineral pigments, organic pigments and pearlescent pigments.

According to a particular embodiment, the composition comprises at least one filler.

According to a particular embodiment, the composition comprises at least one filler and at least one pigment.

### Fillers

The fillers can be mineral or organic, and of any shape, platelet, spherical or oblong.

According to a particular embodiment, the filters are selected from platelet filters, spherical filters and mixtures thereof.

The fillers advantageously have an average diameter greater than 100 nanometers (nm) and less than 200 micrometers (µm), and more particularly a diameter comprised between 5 and 150 µm.

According to a variant implementation of the invention, the filters can be treated so as to modify their surface condition. The fillers used can advantageously be surface-treated to make them hydrophilic, while guaranteeing sufficient cohesion of the powder of the invention. According to a particular embodiment, the filters can be treated in the same way as the aforementioned pigments, with a hydrophilic additive as described above.

The filters are selected in particular from inorganic fillers such as:
talc preferably in the form of particles generally of dimensions less than 40 µm;
micas of natural or synthetic origin, sericites, having dimensions of 2 to 200 µm, preferably of 5 to 70 µm and a thickness of 0.1 to 5 µm, preferably of 0.2 to 3 µm;
kaolin having particle sizes generally less than 30 µm;
metallic soaps derived from organic carboxylic acids having from 8 to 22 carbon atoms, preferably from 12 to 18 carbon atoms, for example zinc stearate, magnesium stearate or lithium stearate, zinc laurate, magnesium myristate, preferably in the form of particles having dimensions of less than 10 µm; these metal soaps play in particular the role of cohesive agent in the powder;
zinc oxides, titanium oxides; calcium carbonate; barium sulphate; magnesium carbonate, magnesium hydrocarbon, silica, glass beads, ceramic beads; boron nitride;
and mixtures thereof.

As organic filters, mention may in particular be made of:
crosslinked or non-crosslinked starches, for example corn, wheat, rice or potato starches;
cellulose powders;
synthetic polymer powders, crosslinked or not, spheronized or not, expanded or not, such as polyamide powders (for example poly-R-alanine powders and nylon powders such as those marketed under the name ORGASOL®), poly(meth)acrylic acid or poly(meth)acylate powders such as cross-linked methyl methacrylate powders, silicone resin powders such as silsesquioxanes,
and mixtures thereof.

According to a particular embodiment, the composition of the invention will comprise at least fillers playing the role of excipient, filters playing a role on the texture and the feel and filters playing a cohesion role within the composition.

Thus the composition of the invention will advantageously comprise:
A filter selected from the group consisting of talc, mica, sericite, synthetic fluorophlogopite, boron nitride, barium sulfate, amino acids, starches, and mixtures thereof, as an excipient agent;
A filler selected from the group consisting of silicas, silicone resin, PMMA, cellulose, and Nylon-12, as texture or feel agent, and
A filler selected from the group of metallic soaps derived from organic carboxylic acids having from 8 to 22 carbon atoms, preferably from 12 to 18 carbon atoms, preferably selected from zinc stearate, magnesium stearate, magnesium myristate and mixtures thereof.

Fillers can also be classified according to their shape. The composition will advantageously comprise spherical fillers and platelet filters.

According to a particular embodiment, the platelet filters are selected from the group consisting of talc, mica, sericite, synthetic fluorphlogopite, boron nitride, barium sulphate, amino acids and mixtures thereof and the spherical filters are selected from the group consisting of silica, PMMA particles, cellulose particles, nylon particles, silicone resin particles, starches and mixtures thereof.

Thus the composition of the invention may advantageously comprise at least:
A filler selected from the group consisting of talc, mica, sericite, synthetic fluorophlogopite, boron nitride, barium sulfate, amino acids, and mixtures thereof, as platelet filler; and
A filter selected from the group consisting of silicas, silicone resin, PMMA, cellulose, and Nylon-12, starches and mixtures thereof as a spherical filler.

The amount of spherical filters and platelet fillers can be adjusted according to the desired properties. In particular, the composition may comprise an amount of from 0.1 to 50%, in particular 1 to 25% by weight, of spherical fillers selected from the group consisting of silica, silicone resin, PMMA, cellulose, Nylon-12, and mixtures thereof, and an amount of from 0.1% to 70%, in particular 1% to 60%, of platelet filters.

According to a particular and preferred embodiment, the composition also comprises at least one filler selected from metallic soaps derived from organic carboxylic acids having from 8 to 22 carbon atoms, in an amount of from 0% to 10% by weight with respect to the total weight of said composition, to improve the cohesion of the powder obtained according to the invention.

### Advantageously Hydrophilic Treated Pigments

According to one particular embodiment, in particular when the composition of the invention is a makeup composition, it comprises at least one dyestuff, preferably at least one pigment. The pigment can be selected from mineral pigments, organic pigments, pearlescent pigments and mixtures thereof. Preferably, the composition comprises at least mineral pigments.

The mineral pigments can be selected from iron oxides, in particular black, yellow, red and brown iron oxides; titanium dioxide, manganese violet; ultramarine blue; chromium oxides, in particular hydrated chromium oxide, ferric blue, carbon black, and mixtures thereof.

Among the organic pigments, mention may in particular be made of the lakes obtained from dyes such as the dyes D&C Black No. 2, FD&C Blue No. 1, FD&C Green No. 3, D&C Green No. 5, D&C Orange No. 4, D&C Orange No. 5, D&C orange No. 10, D&C No. red 3, D&C Red No. 6, D&C Red No. 7, D&C red No. 9, D&C red No. 13, D&C red No. 19, D&C Red No. 21, D&C Red No. 22, D&C Red No. 27, D&C Red No. 28, D&C Red No. 30, D&C Red No. 33, D&C Red No. 36, FD&C Red No. 40, FD&C Yellow No. 5, FD&C Yellow No. 6, D&C Yellow No. 10 and Cochineal Carmine.

The pearlescent pigments are for example selected from mica covered with titanium oxide, mica-titanium covered with iron oxide, mica-titanium covered with ferric blue, mica-titanium covered with chromium oxide, mica-titanium covered with an organic pigment as described previously, as well as pigments based on bismuth oxychloride.

The pigments used in the composition of the invention are advantageously treated with a hydrophilic additive to facilitate their dispersion in the composition.

By way of hydrophilic additive, mention may in particular be made of phytic acid, alginate and sodium glycerophosphate (SGP). Preferably, a hydrophilic additive from those described below will be used.

According to a first particular embodiment, the pigments used according to the invention are pigments treated with sodium glycerophosphate (otherwise called ‘SGP’ pigments), in particular iron oxides treated with such an additive.

Such pigments are described in particular in the international application WO2012120098 from SENSIENT.

Thus, according to a particular embodiment, the composition of the invention will comprise pigments or a pigmentary composition comprising a pigment and an additive of formula (I)

### [Chem 1]

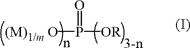

in which:
n represents 1 or 2,
M represents H or a cation,
m represents 1 when M is H and m represents the valence of the cation when M is a cation,
R represents:
a group G selected from a saccharide or a group —[CH₂—CHR₁—O]_{q}—R₂ or —[CH₂—CH(CH₂OH)—O]_{q}—R₂ where:
q represents an integer from 1 to 1000,
for each CH₂-CHR₁-O unit, R1 independently represents H or a methyl,
R₂ represents H or an alkyl comprising from 1 to 3 carbon atoms, and,
a hydrocarbon chain comprising from 1 to 500 carbon atoms substituted by one or more G, phosphate (of formula OPO3(M)_{2/m}) and/or hydroxyl (OH) groups.

This pigmentary composition is suitable for introduction into a cosmetic composition, in particular aqueous cosmetic composition. It is generally in the form of a powder comprising the pigment and the additive intimately mixed, the additive generally being adsorbed or precipitated on the surface of the pigment. A pigmentary composition in powder form is particularly advantageous compared to a composition in liquid form (suspension, emulsion or solution).

It turns out that the pigment compositions described in application WO2012120098 disperse very easily and effectively in an aqueous medium or in the continuous aqueous phase of an oil-in-water emulsion.

In this compound of formula (I), it is meant that the hydrocarbon chain comprises from 1 to 500 carbon atoms, in particular from 1 to 50, typically from 1 to 10 carbon atoms, preferably from 1 to 5 carbon atoms. Hydrocarbon chains can be linear, branched or cyclic. The preferred hydrocarbon chains are alkyl groups (preferably having 1 to 10 carbon atoms, in particular 1 to 5 carbon atoms, preferably 1 to 3, such as methyl, ethyl, n-propyl and isopropyl groups), alkenyl (preferably having 2 to 10 carbon atoms, in particular 2 to 6), aryl (preferably having 6 to 10 carbon atoms), arylalkyl (preferably having 7 to 10 carbon atoms) or alkylaryl (preferably having 7 to 10 carbon atoms). M can in particular be an inorganic cation, such as Ag³⁺, Al³⁺, Fe³⁺, Fe²⁺, Ag²⁺, Zn²⁺, Sn²⁺, Ca²⁺, Ba²⁺, Ag⁺, Na⁺ or an organic cation, such as a diethanolammonium (DEA) (H₃N⁺-(CH₂)₂-OH) or a quaternary ammonium.

According to a particular embodiment, the pigment or pigmentary composition comprises an additive of formula (II) below:

### [Chem 2]

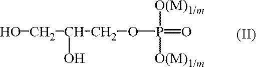

In which M and m are as defined above, (which corresponds to an additive of formula (I) in which n represents 2, R represents a group G of formula —[CH₂—CH(CH₂OH)—O]_{q}—R₂ where q represents 1 and R₂ represents H.

According to a particular and preferred embodiment, a glycerophosphate additive of the following formula (III) is used: [Chem 3]

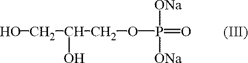

which corresponds to an additive of formula (II) in which M represents Na and m represents 1, this additive being advantageously commercially available.

Thus, according to a particular and preferred embodiment, the pigments used according to the invention are pigments treated with sodium glycerophosphate (otherwise called ‘SGP’ pigments), in particular iron oxides treated with such an additive.

Mention may in particular be made of the pigments treated with sodium glycerophosphate (SGP) from SENSIENT marketed according to the following references:
o Unipure red LC381 SGP (iron oxides, CI77491 and CI 77499 and sodium glycerophosphate)
o Unipure black LC989 SGP (iron oxides, CI 77499 and alumina and magnesium oxide and sodium glycerophosphate)
o Unipure yellow LC182 SGP (iron oxides, CI 77492 and sodium glycerophosphate)
o Unipure white LC 981 SGP (titanium dioxide, CI 77891 and sodium glycerophosphate).

According to another particular embodiment, an additive of formula (V) is used [Chem 4]

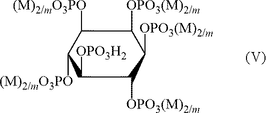

which corresponds to an additive of formula (I) in which n represents 2, R represents a cyclohexyl hydrocarbon chain substituted in positions 2, 3, 4, 5 and 6 by a phosphate group of formula OPO₃H₂. And preferably phytic acid of formula (V′) [Chem 5]

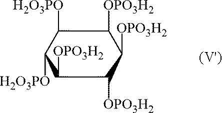

Which corresponds to an additive of formula (V) in which M represents H and m represents 1, this additive being advantageously commercially available, for example from Nutriscience®.

The pigments treated with a phytic acid solution of formula (V′) are described in application WO2012/120098 and FR3080116 from Sensient.

Mention may in particular be made of the pigments treated with a phytic acid (PHY) from SENSIENT marketed according to the following commercial references:
o Unipure red LC388 PHY (red iron oxide, CI 77491 and phytic acid and sodium hydroxide)
o Unipure black LC 998 PHY (CI 77499 and phytic acid and sodium glycerophosphate)
o Unipure yellow LC 188 PHY (CI 77492 and phytic acid and sodium glycerophosphate)
o Unipure white LC 985 PHY (anastase, CI 77891 and phytic acid and sodium glycerophosphate).

According to a particular embodiment of the invention, the composition of the invention will comprise pigments treated with an additive of formula (I), (II), (III) or (V), preferably a sodium glycerophosphate additive of formula (III) or a phytic acid additive of formula (V′).

According to another particular embodiment, the pigments are treated with an alginate (INCI name: algin). Alginate means alginate or one of its salts, in particular the sodium salt.

This anionic polysaccharide is found naturally in oceans around the world in the cell walls of brown algae and kelp. Inorganic pigments such as iron oxide and titanium dioxide treated with sodium alginate are highly hydrophilic and provide excellent dispersibility and redispersibility in the aqueous phase. Such pigments treated with a sodium alginate are marketed under the name MiyoAQUA^{ⓒ} by the company Miyoshi America.

Mention may in particular be made of pigments treated with an alginate under the name MiyoAQUA^{ⓒ} from Miyoshi America:
o MiyoAQUA^{ⓒ} RED (CI 77491 and alginate)
o MiyoAQUA^{ⓒ} BLACK (CI 77499 and alginate)
o MiyoAQUA^{ⓒ} YELLOW (CI 77492 and alginate)
o MiyoAQUA^{ⓒ} WHITE (titanium dioxide or CI 77891 and alginate)

The treated or untreated pigments may be present in an amount of from 0.1% to 60%, in particular from 0.1% to 50% and preferably from 0.1% to 30% by weight relative to the total weight of said composition. For makeup compositions, the amount of treated or untreated pigment(s) will generally range from 10% to 25% by weight, in particular from 10% to 20% by weight relative to the total weight of said composition.

### Wet Preparation Method (formation of an Intermediate Paste ‘Slurry’)

The solid fresh and hydrating composition of the invention can be produced by the wet method by preparing a paste (or pasty composition) according to a suitable industrial method.

According to one of its aspects, the object of the invention is a method for manufacturing the solid fresh and hydrating composition described above, which method comprises: i) mixing the particulate phase, and the hydroxyl compound, the water and the optional oily phase constituting the liquid phase, to form a paste; and (ii) shaping the paste, in particular in a silicone mold, by compacting and partial removal of the liquid phase present in the paste.

The liquid phase consists of water and any additional volatile or non-volatile solvents and binders (oils).

‘Partial removal’ according to the invention, means that the solid hydrating composition obtained comprises a final rate of water sufficient to give it the desired hydrating properties. Thus the solid hydrating composition will comprise an amount of 5% to 50% of water relative to the total weight of said composition. According to a particular embodiment, the partial removal is done by vacuum suction, at room temperature and in particular with a suction pressure between 1 and 4 bars, a compression pressure between 1 and 4 bars (limits included).

Thanks to the presence of water in the slurry method, it is possible to incorporate in the composition suspending agents which will allow to preserve the paste for a certain time in storage phase so that the pigments remain suspended, that there is no release and that the paste remains homogeneous.

As suspending agents, and as described above, use can advantageously be made of magnesium aluminum silicate, hectorite, bentonite, xanthan gum, chondrus crispus and mixtures thereof.

Thus, a composition of the invention will also comprise at least one suspending agent, in particular selected from magnesium aluminum silicate, hectorite, bentonite, xanthan gum, chondrus crispus and mixtures thereof.

The method of the present invention, using water and a particulate phase comprising fillers and optionally pigments, which are advantageously hydrophilic treated, advantageously allows to obtain a homogeneous paste and homogeneous compact powders. “Homogeneous” means a composition in which the various constituents and in particular the pigments, when they are present, are distributed to give a uniform or substantially uniform appearance to the naked eye. In particular, the composition obtained according to the method of the invention, does not have surface color irregularities, such as marbling.

According to another of its aspects, the object of the invention is a method for manufacturing a solid fresh and hydrating composition, which method comprises: (i) mixing the particulate phase, and the hydroxyl compound, the water and the optional oily phase constituting the liquid phase, so as to form a paste; and (ii) shaping the paste, in particular in a silicone mold, by compacting and partial removal of the liquid phase (volatile or non-volatile, for example water) present in the paste, in particular by vacuum suction, optionally followed by freezing, and optionally drying at a temperature below 70° C.

It is implicit that at the time of the partial removal of the water, part of the oily (volatile or non-volatile) phase can also be removed.

‘Partial removal’ according to the invention, means that the solid composition obtained comprises a residual water rate, preferably at least 5% by weight, in the final product. The partial removal is carried out, according to a particular embodiment, by suction under vacuum at ambient temperature and with a suction pressure between 1 and 4 bars, a compression pressure between 1 and 4 bars.

The paste (‘slurry’) is obtained by mixing the particulate solid phase, the hydroxyl compound selected from polyols, glycol ethers and mixtures thereof, water, and the optional oily phase.

The paste comprises the various constituents, preferably in the following proportions:
from 15% to 80% by weight of particulate solid phase relative to the total weight of the paste;
10 to 70% by weight of water relative to the total weight of the paste; and
0.1 to 30% by weight of oils and/or structuring agents relative to the total weight of the paste.

The paste can be shaped or not.

For shaping, the paste can be placed in molds in order to undergo shaping according to a technique known to the person skilled in the art.

The molds can be made of metal or silicone and can be reused once the composition has been removed from the mold. The molds can also be cups or buckets which remain attached to the composition once manufactured in order to then be placed in the housing of a packaging. When the paste is liquid, it can be injected through the bottom, the side or the top of the mold or be poured by simply filling the mold through its upper opening. A method for injection through the bottom of the molds is for example known under the name “Back Injection Machine” or BIM. A method for injection from the side is for example known by the name “Side Injection”. A method for filling molds that involves pouring the paste into the mold from above is known as “Top Fill”. The paste can also be more viscous so that it can be dosed by preparing pieces of paste. The percentage of volatile or non-volatile liquid (water and optional solvents and additional volatile or non-volatile binders) is preferably selected such that the paste has a sufficient consistency to be able to be kneaded, measured and cut. In this case, it is necessary to shape the paste so that the composition hugs the edges of the buckets, before partially removing the volatile or non-volatile liquid. This shaping can be carried out by pressing (compacting) at the same time as the partial removal of the volatile or non-volatile liquid.

The preparation method of the invention comprises a step of partially removing the volatile or non-volatile liquid, the purpose of which is to partially remove the volatile or non-volatile liquid in order to solidify the composition while maintaining a sufficient rate of water to keep the smoothness of the composition and to give it a creamy appearance with hydrating properties, despite a solid appearance.

The partial removal of the volatile or non-volatile liquid can be carried out with mechanical means such as suction, or thermal means such as heat. According to a particular and preferred embodiment, the partial removal of the volatile or non-volatile liquid is done by suction under vacuum, at room temperature and with a suction pressure preferably between 1 and 4 bars, a compression pressure preferably between 1 and 4 bars (limits included).

The person skilled in the art will be able to adapt the duration of this step to optimize the partial removal of the volatile or non-volatile liquid. The paste may comprise a higher or lower proportion of volatile or non-volatile liquid depending on the duration and the means of removal used. According to the invention, the paste will comprise a high proportion of water as described above to give it its hydrating properties.

The water thus preferably represents from 10% to 70% by weight of the paste, and from 5% to 50% by weight of the final solid composition according to the invention.

The pressing (compacting) is carried out in particular by means of a stamp which contacts the paste and which compresses the composition between the bottom of the mold and its surface during suction. The end of the stamp or the bottom of the mold is perforated to allow the extraction of volatile or non-volatile liquid out of the paste during suction.

In this embodiment, a porous material can be deposited on the free surface of the paste in each mold, or be placed on the stamp which serves as a press in order to retain the powder in the mold during suction. This porous material is for example a fabric, a weft or an absorbent sheet. It is removed from the composition once the suction is complete. The partial removal of the volatile or non-volatile liquid can also comprise the combination of the means described above. Thus, in accordance with a variant of the method of the invention, the partial removal of the volatile or non-volatile liquid contained in the paste comprises a mechanical removal (vacuum suction) followed or not by thermal removal.

The method can comprise for example two successive steps: partially drying the paste using mechanical removal means to obtain a wet powder, and partially drying the wet solid composition by thermal means. The mechanical partial removal can be carried out by suction under a press, and the thermal partial removal can be carried out for example by drying in an oven, preferably at a temperature below 70° C., more preferably still below 60° C.

According to a particular and preferred embodiment, the partial removal of the volatile or non-volatile liquid is done by suction under vacuum, at room temperature and with a suction pressure preferably between 1 and 4 bars, a compression pressure preferably between 1 and 4 bars (terminals included).

According to a first preferred embodiment, the compositions of the invention are prepared by a method which comprises:
the preparation of the paste (‘slurry’),
introducing the paste into a mold, for example by means of a pump and an injection unit which advantageously comprises nozzles allowing the paste to be injected from below or above the molds,
depositing a porous material on the free surface of the paste and/or depositing a plastic grid on the surface before depositing the porous material,
vacuuming the filled molds and partially removing by suction the volatile or non-volatile liquid present in the paste, preferably by maintaining the paste under a press during the suction, and
returning to atmospheric pressure.

The present invention is particularly suitable for the preparation of cosmetic compositions intended for caring for or making up the body and/or the face. The method of the invention allows to prepare a solid powder of homogeneous appearance, in which the various components and in particular the particulate phase is well dispersed.

### Method in Buckets

According to a first embodiment, called ‘1st generation’ embodiment (1G method), the paste is compacted from above in a bucket.

During compaction, compression and suction take place simultaneously using tools with a perforated head and a compaction pattern.

The compaction fabric is in contact with the pattern. The rest of the solvent is evaporated in an oven for several hours.

Compression is carried out in two steps:
the first compaction allows to partially suck up the solvent
the second compaction gives the final shape with patterns on the surface.

### Silicone Mold Method

According to a second embodiment, called 2^{nd} generation embodiment (2G method), the solid hydrating composition obtained no longer needs a bucket, but uses silicone molds and has a 3-dimensional pattern (3D pattern) which is more precise than with the previous embodiment. The visuals and reliefs (3D effect, precision, gestures) are new. The 2G method allows to achieve surface volumes greater than 5 mm, much larger than with conventional compaction, as well as a beautiful surface appearance, good pattern definition and gloss. In addition, the surface appearance of the 2G method is much more precise and brighter than the 1G method due to the absence of intermediate compacting during the creation of the relief (pattern present in the silicone mold).

This 2G method also allows to incorporate a high rate of binder compared to a powder formula produced by dry compaction, and to incorporate a high percentage of water in a solid composition, for example more than 20% by weight of the composition, to increase the feeling of freshness and hydration when applied.

### Preparation of the Paste According to the 2G Method

The paste is made by pasting the powders with the binder and the solvent. The mixing of the phases is carried out using a Rayneri and a blade of the deflocculating type.

The water, the suspending agents (for example bentonite and/or hectorite gels), the preservatives, the glycols and any additional oils are homogenized with stirring then the particulate solid phase (powder phase) is gradually incorporated into the rest of the mixture to thereby form a paste, called a paste or slurry. In the case of adding volatile oils, they are incorporated after the addition of the powder phase, at the end.

### Shaping the Powder According to the 2G Method

For shaping the solid hydrating composition, use is advantageously made of silicone molds. These molds allow to create new surface reliefs and patterns. These reliefs and patterns are represented in negative on the internal face of the mold.

The paste is incorporated into the silicone molds, a support grid is added on the surface to improve the solidity of the compact.

Then a compacting fabric is added then the compaction is carried out from the top of the mold and the solvent is partially sucked up using a perforated smooth surface tool.

Compaction is carried out, for example, using a Top Fill, Back Fill, or Side Injection machine.

During compression, the fabric is no longer in contact with the pattern, thus the pattern created on the surface is more precise and more in relief compared to the method with a bucket.

After compacting, the paste is placed in the freezer in their silicone molds for a duration between 15 minutes and 2 hours to facilitate demolding and to optimize the definition of the design during demolding.

Then, they are optionally placed in an oven at 45° C. or 50° C. for a more or less long period depending on the amount of solvent (water) that it is desired to keep in the end in the final product.

For an amount between 5 and 50% by weight of water in the final product, the duration may range from a few minutes to 6 hours depending on the means used (furnace, oven at 45° C. or 50° C. or no drying), to modulate the quantity of water in the final product.

Thus, according to a particular embodiment, the compositions of the invention are prepared according to the following method, illustrated in the examples below, according to the following steps:
(i) mixing a liquid phase comprising at least water and the solid phase in powder form, to form a paste (‘slurry’):
   a. the ingredients of the aqueous phase A are dissolved in an excess quantity of water such that phase A is approximately equivalent in weight to the particulate solid phase B;
   b. the ingredients of phase B are mixed then dispersed in phase A using a Rayneri; for example at 1000 revolutions/minute for 5 to 10 minutes until a homogeneous mixture is obtained;
   c. the optional binding phase C is added last to the pasty dispersion formed by phases A and B. The slurry is homogenized, for example at 1000 revolutions/minute for 15 minutes;
(ii) shaping the paste by compacting and partial removal of the liquid phase so as to give the composition in the form of a compact powder, according to the following steps:
   a. the paste is incorporated into molds, for example silicone molds, taking care to remove any air bubbles; advantageously a support grid is added on the surface to improve the solidity of the compact;
   b. a compacting fabric is added then compacting is carried out, for example, from above the mold (Top Fill machine) and the solvent (water) is simultaneously sucked up by creating a vacuum in the enclosure (vacuum suction at ambient temperature) with a compression pressure of 1 to 4 bar and a suction pressure of 1 to 4 bar); the fabric no longer being in contact with the pattern in this step, the pattern created on the surface is more precise and more in relief compared to the 1G method;
   c. the molds are then advantageously placed in the freezer for 30 minutes to 1 hour to allow easy unmolding;
   d. after unmolding, and if necessary, the products are placed in an oven at 50° C. for 5 hours or alternatively at 45° C. overnight until the water amount that is desired to be retained in the final product is reached.

### Galenic

According to one particular embodiment, the composition of the invention is in the form of a solid hydrating care composition or a solid hydrating composition for making up the face and/or the lips, in particular a blush, an eye shadow, a highlighter, a bronzing powder, a protective foundation powder, a concealer, a lip powder or an eyebrow powder. The composition of the invention is preferably a highlighter, a solid hydrating foundation composition, an eye shadow, a blush or a lipstick.

According to a particular and preferred embodiment, the composition of the invention has three-dimensional (3D) relief patterns on the surface, bringing out a particular pattern such as a logo, an alphanumeric character or a string of alphanumeric characters, or a representation styled with an object such as a flower.

This aspect in relief, is visually very attractive, is made possible thanks to the use of silicone molds adapted to the desired shape, the use of which is very suitable for the shaping method used for the solid composition of the compact powder type of the invention.

The application of the solid hydrating compositions according to the invention can be carried out with a brush, sponge or finger.

### Cosmetic Method

The invention also concerns a cosmetic method for caring for and/or making up keratin materials, more particularly the skin, comprising the application to the keratin materials of a composition according to the invention.

Applied to the skin, in particular of the face, the solid fresh and hydrating composition, in particular obtained by a ‘slurry’ method as described above, provides the consumer with the comfort and sensoriality sought, with a good make-up result.

The invention will now be illustrated in the following non-limiting examples. Unless otherwise indicated, the % are expressed as % by weight of raw material relative to the total weight of the composition.

### EXAMPLES

In the following examples, the terms “SGP TREATED PIGMENTS” and “SGP TREATED PIGMENTS” respectively designate mineral pigments which have received a hydrophilic surface treatment with sodium glycerophosphate (SGP) or phytic acid (PHY), giving said pigments an affinity for hydrophilic media such as an aqueous phase. Their use allows to surprisingly obtain a slurry of powders whose unctuous and fresh texture is particularly pleasant when applied, while retaining its properties of homogeneity and hardness after shaping the slurry in the form of a compact. These treated pigments are, for example, marketed under the brand UNIPURE® by the company SENSIENT COSMETIC TECHNOLOGIES.

The term “ALG-TREATED PIGMENTS” designates mineral pigments that have received a hydrophilic surface treatment with Alginate (ALG). These treated pigments are for example marketed under the brand MiyoAQUA^{ⓒ} by the company Miyoshi America.

In accordance with the method of the invention, in the following examples; the aqueous phase A comprises an excess of water, at least part of which is removed at the time of the solvent (volatile or non-volatile liquid) suction step.

Moreover, the initial quantity of water must therefore be calculated to obtain the intermediate water percentage indicated at the end of the slurry preparation step, formed by mixing phases A and B, and optionally phase C.

The removal of the solvent is controlled so as to obtain the final percentage of water indicated in the tables below (final compositions).

### Example 1 Hydrating Care Powder

A care powder according to the invention and a control which differs from the invention in that it does not contain glycerol, are prepared.

The final formulas of the compositions (Invention and Control) are presented in Table 1 below (% by weight of the final composition).

TABLE 1

| Phase | Ingredients | Control (% by weight) | Example 1 according to the invention (% by weight) |
|---|---|---|---|
| A | WATER | 43 | 37 |
| A | GLYCEROL | -- | 9.8 |
| A | HECTORITE (BENTONE®) | 0.1 | 0.1 |
| A | MAGNESIUM ALUMINUM SILICATE (VEEGUM® HV) | 0.1 | 0.1 |
| A | PHENOXYETHANOL | 0.5 | 0.6 |
| A | CAPRYLYL GLYCOL | 0.2 | 0.2 |
| B | FILLERS | 51.6 | 45.4 |
| B | TITANIUM DIOXIDE (AND) SODIUM GLYCEROPHOSPHATE (UNIPURE® SGP) | 4.5 | 6.8 |

Phase A: aqueous phase
Phase B: particulate phase (powders)

To prepare one or the other of the final formulas presented in the Table above, the ingredients of Phase A comprising an excess of water are mixed together, which are cold dispersed with a Rayneri at 500 revolutions/minute for 5-10 minutes. The mixture of powders from phase B is then added to phase A, then the whole is dispersed with a Rayneri, gradually increasing the speed to a maximum of 1200 revolutions/minute to obtain a visually homogeneous mixture.

At this stage, the water is present in excess in the slurry, in a quantity of 132% by weight relative to the sum of all the other ingredients of the mixture for the control formula, and 107% for the composition of the invention.

The shaping is carried out by filling, in Top Fill mode, silicone molds equipped with a support grid. A fabric is placed on the surface, then the solvent of the powder slurry is sucked off at 4 bar for 30 seconds.

The mold is placed in the freezer for 1 hour.

After unmolding, the composition is placed in an oven at 45° C. for two hours.

After this drying step, the composition is placed in a sealed pack.

### Hydration Test

The hydration is measured by corneometry, using a CORNEOMETER CM825 apparatus supplied by the company Courage & Khazaka SN “NUM_SERIE”, which with the person skilled in the art is familiar.

The method for measuring the hydration of the stratum corneum is based on the creation of an electric field on the surface of the skin and the detection, using electrodes, of variations in the dielectric constant induced by the state of hydration of the upper layer of the epidermis (see Measurement of the electrical properties is recognized as an objective method of assessing stratum corneum hydration (E. Berardesca - EEMCO guidance for the assessment of stratum corneum hydration: electrical methods - Skin Research and Technology 1997; 3:126-132).

The control formula or the composition of the invention is applied to the forearm of a volunteer. The quantity applied corresponds to 2 µl/cm² of skin. The application area is then protected from friction by placing a metal ring surrounding the application area, fixed to the forearm by adhesive.

A measurement is taken on the application area, before application of the composition, then 6 hours after said application of the composition, leaving a period of 15 minutes for stabilization after removal of the anti-friction protection, before the measurement.

The residual film of the control formula or of the composition of the invention is wiped off from the area of application.

The variation in hydration is calculated from the measurements carried out according to the method described previously.

The results are shown in Table 2 below:

TABLE 2

| | Control composition | Example 1 according to the invention |
|---|---|---|
| Hydration at T 6h | < 5% | + 27% |

The composition of the invention (water + hydroxyl compound) allows important and significant hydration of the skin, whereas the control formula (water) only preserves the initial hydration of the skin after application of the formula to the test area.

### Example 2 Pearl Bronzing Powder

A compact powder is prepared according to the final formula shown in Table 3 below:

TABLE 3

| Phase | Ingredients | Example 2 according to the invention (% by weight) |
|---|---|---|
| A | WATER | 31 |
| | GLYCEROL | 10.7 |
| A | HECTORITE (BENTONE®) | 0.1 |
| A | MAGNESIUM ALUMINUM SILICATE (VEEGUM ® HV) | 0.1 |
| A | PHENOXYETHANOL | 0.6 |
| A | CAPRYLYL GLYCOL | 0.3 |
| B | PEARLS | 40.3 |
| B | LAUROYL LYSIN | 1.7 |
| B | CELLULOSE (CELLULOBEAD®) | 2.4 |
| B | IRON OXIDE (AND) SODIUM GLYCEROPHOSPHATE (UNIPURE® SGP) | 4.4 |
| B | TITANIUM DIOXIDE (AND) SILICA (SPHERICA HC WHITE 30) | 6.3 |
| C | UNDECANE and TRIDECANE (CETIOL® ULTIMATE MB) | 2.1 |

Phase A: aqueous phase
Phase B: particulate phase (powders)
Phase C: oily phase

To prepare the compact powder presented above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with a Rayneri, gradually increasing the speed to a maximum of 1200 revolutions/minute.

At this stage, the water is present in excess in the slurry, in a quantity of 83% by weight compared to the sum of all the other ingredients of the mixture.

Phase C is added last while continuing to homogenize the paste.

The shaping is carried out by filling, in Top Fill mode, silicone molds equipped with a support grid. A fabric is placed, then a step of compression (Pressure = 2 bars) and suction of the solvent (Pressure = 1 bar) is carried out for 20 seconds

The mold is placed in the freezer for 1 hour.

After demolding, the composition is placed in a sealed pack.

### Hardness Measurement

The hardness is measured using a TA.XT PLUS TEXTURE ANALYSER (STABLE MICROSYSTEMS) texturometer, equipped with a rod on which is fixed a cylinder with a diameter of 5 mm which is applied to the surface of the compact. The cylinder penetration force is measured over a thickness of 1 mm at a speed of 0.5 mm/second.

The average hardness, measured on three parts, according to the protocol previously described, is equal to 279 grams force (Gf).

### Staying Power

The power of the composition for staying on the skin is evaluated by measuring the residual film, 6 hours then 8 hours after its application to the skin.

The foundation of the example has a residual film of 91% after 6 hours and 84% after 8 hours.

### Hydration Test

During the hydration test carried out according to the protocol of Example 1, the composition showed a significant hydrating effect of +18% relative to the control after 6 hours of application to the skin.

### Example 3 Matte Bronzing Powder

A tinted powder for making up the skin is prepared according to the final formula presented in Table 4 below:

TABLE 4

| Phase | Ingredients | Example 3 according to the invention (% by weight) |
|---|---|---|
| A | WATER | 35 |
| | GLYCEROL | 10.1 |
| A | HECTORITE (BENTONE®) | 0.1 |
| A | MAGNESIUM ALUMINUM SILICATE (VEEGUM® HV ) | 0.1 |
| A | PHENOXYETHANOL | 0.6 |
| A | CAPRYLYL GLYCOL | 0.3 |
| B | PEARLS | 32.9 |
| B | LAUROYL LYSIN | 6.5 |
| B | CELLULOSE (CELLULOBEAD®) | 2.3 |
| B | IRON OXIDE (AND) SODIUM GLYCEROPHOSPHATE (UNIPURE® SGP) | 5.1 |
| B | TITANIUM DIOXIDE (AND) SILICA (SPHERICA HC WHITE 30) | 3.8 |
| C | UNDECANE and TRIDECANE (CETIOL® ULTIMATE MB) | 3.2 |

Phase A: aqueous phase
Phase B: particulate phase (powders)
Phase C: oily phase

To prepare the composition shown in the table above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with a Rayneri, gradually increasing the speed to a maximum of 1200 revolutions/minute.

Phase C is added last while continuing to homogenize the paste.

At this stage, the water is present in excess in the slurry, in a quantity of 107% by weight compared to the sum of all the other ingredients of the mixture.

The shaping is carried out by filling, in Top Fill mode, silicone molds equipped with a support grid. A fabric is placed, then a step of compression (Pressure = 2 bars) and suction of the solvent (Pressure = 2 bars) is carried out for 20 seconds.

The mold is placed in the freezer for 1 hour.

After demolding, the composition is placed in a sealed pack.

### Hardness Measurement

The hardness is measured according to the protocol of Example 2.

The average hardness of the composition, measured on three pieces of the composition prepared above, is equal to 271 grams force (Gf).

### Hydration Test

During the hydration test carried out according to the protocol of Example 1, the composition of this Example 3 according to the invention showed a significant hydrating effect of +10% relative to the control after 6 hours of application to the skin.

### Example 4 Highlighter

The solid composition is prepared according to the final formula shown in Table 5 below (% by weight of the final composition):

TABLE 5

| Phase | Ingredients | Example 4 according to the invention (% by weight) |
|---|---|---|
| A | WATER | 31 |
| | GLYCEROL | 10.7 |
| A | HECTORITE (BENTONE®) | 0.1 |
| A | MAGNESIUM ALUMINUM SILICATE (VEEGUM® HV ) | 0.1 |
| A | PHENOXYETHANOL | 0.7 |
| A | CAPRYLYL GLYCOL | 0.3 |
| B | PEARLS | 39.1 |
| B | LAUROYL LYSIN | 1.4 |
| B | CELLULOSE (CELLULOBEAD®) | 2.4 |
| B | IRON OXIDE (AND) SODIUM GLYCEROPHOSPHATE (UNIPURE® SGP) | 0.2 |
| B | TITANIUM DIOXIDE (AND) SILICA (SPHERICA HC WHITE 30) | 7.3 |
| B | CALCIUM ALUMINUM BOROSILICATE | 4.6 |
| C | UNDECANE and TRIDECANE (CETIOL® ULTIMATE MB) | 2.1 |

Phase A: aqueous phase
Phase B: particulate phase (powders)
Phase C: oily phase

To prepare the composition shown in the table above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with a Rayneri, gradually increasing the speed to a maximum of 1200 revolutions/minute.

Phase C is added last while continuing to homogenize the paste.

At this stage, the water is present in excess in the slurry, in a quantity of 74% by weight compared to the sum of all the other ingredients of the mixture.

The shaping is carried out by filling, in Top Fill mode, silicone molds equipped with a support grid. A fabric is placed on the surface of the formula, then a step of compression (Pressure = 2 bars) and suction of the solvent (Pressure = 2 bars) is carried out for 20 seconds.

The mold is placed in the freezer for 1 hour.

After demolding, the composition is placed in an oven at 45° C. for one hour.

After the drying step, the composition is placed in a sealed pack.

### Hardness Measurement

The hardness is measured according to the protocol of Example 2.

The average hardness of the composition of this Example 4, measured on two pieces, according to the protocol previously described, is equal to 288 grams force (Gf).

### Hydration Test

During the hydration test carried out according to the protocol of Example 1, the composition of this Example 4 according to the invention showed a significant hydrating effect of +12% relative to the control after 6 hours of application to the skin.

The highlighter also has an attractive three-dimensional visual effect, the surface reliefs and patterns of which have particularly well-defined contours.

### Example 5 Matte Foundation

A solid foundation having a matte finish after application to the skin is prepared.

The formula of the composition is presented in Table 6 below:

TABLE 6

| Phase | Ingredients | % by weight |
|---|---|---|
| A | WATER | 33.3 |
| | GLYCEROL | 10.3 |
| A | HECTORITE (BENTONE®) | 0.1 |
| A | MAGNESIUM ALUMINUM SILICATE (VEEGUM ® HV) | 0.1 |
| A | PHENOXYETHANOL | 0.6 |
| A | CAPRYLYL GLYCOL | 0.3 |
| B | MICA (MEARLMICA TREAT SVA) | 32.2 |
| B | LAUROYL LYSIN | 6.7 |
| B | CELLULOSE (CELLULOBEAD®) | 2.3 |
| B | IRON OXIDE (AND) ALGINATE (MlYOAQUA®) | 2.3 |
| B | TITANIUM DIOXIDE (AND) ALGINATE (MIYOAQUA®) | 3 |
| B | PEARLS | 5.5 |
| C | UNDECANE and TRIDECANE (CETIOL® ULTIMATE MB) | 3.3 |

Phase A: aqueous phase
Phase B: particulate phase (powders)
Phase C: oily phase

To prepare the foundation presented in the table above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with a Rayneri, gradually increasing the speed to a maximum of 1200 revolutions/minute to obtain a visually homogeneous mixture.

Phase C is added last while continuing to homogenize the paste.

At this stage, the water is present in excess in the slurry, in a quantity of 107% by weight compared to the sum of all the other ingredients of the mixture.

The shaping is carried out by filling, in Top Fill mode, silicone molds equipped with a support grid. A fabric is placed, then a step of compression (Pressure = 4 bars) and suction of the solvent (Pressure = 3 bars) is carried out for 40 seconds. The mold is placed in the freezer for 1 hour.

After demolding, the composition is placed in an oven at 45° C. for one hour.

After drying, the composition is placed in a sealed pack

### Example 6 Hydrating Foundation

A foundation for caring for the skin is prepared.

The final formula of the composition is shown in Table 7 below:

TABLE 7

| Phase | Ingredients | Example 6 according to the invention (% by weight) |
|---|---|---|
| A | WATER | 39 |
| | GLYCEROL | 9.5 |
| A | HECTORITE (BENTONE®) | 0.1 |
| A | MAGNESIUM ALUMINUM SILICATE (VEEGUM® HV ) | 0.1 |
| A | PHENOXYETHANOL | 0.6 |
| A | CAPRYLYL GLYCOL | 0.2 |
| B | PEARLS | 35 |
| B | LAUROYL LYSIN | 1.2 |
| B | CELLULOSE (CELLULOBEADO) | 2.1 |
| B | TITANIUM DIOXIDE (AND) SILICA (SPHERICA HC WHITE 30) | 5.4 |
| B | TITANIUM DIOXIDE (AND) ALUMINA (AND) MAGNESIUM OXIDE (AND) SODIUM GLYCEROPHOSPHATE (UNIPURE® SGP) | 6.8 |

Phase A: aqueous phase
Phase B: particulate phase (powders)

To prepare the foundation presented in the table above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with Rayneri, gradually increasing the speed to a maximum of 1200 revolutions/minute and homogenization of the slurry.

At this stage, the water is present in excess in the slurry, in a quantity of 86% by weight compared to the sum of all the other ingredients of the mixture.

The shaping is carried out by filling, in Top Fill mode, silicone molds equipped with a support grid. A fabric is placed then the solvent of the powder slurry is sucked off at 4 bars for 1 minute.

The mold is placed in the freezer for 1 hour. After demolding, the composition is placed in an oven at 45° C. for one hour.

After drying, the composition is placed in a waterproof pack of the “cushion” type.

### Hydration Test

During the hydration test carried out according to the protocol of Example 1, the composition of this Example 6 according to the invention showed a significant hydrating effect of +30% compared to the control after 6 hours of application to the skin.

The foundation also produces when applied an immediate effect of freshness complementary to the hydrating effect of the skin observed at a distance from the application.

### Example 7 Pearly Foundation

A foundation having a pearly effect on the skin is prepared. The formula of the composition is presented in Table 8 below:

TABLE 8

| Phase | Ingredients | Example 2 according to the invention (% by weight) |
|---|---|---|
| A | WATER | 31 |
| | GLYCEROL | 10.7 |
| A | HECTORITE (BENTONE®) | 0.1 |
| A | MAGNESIUM ALUMINUM SILICATE (VEEGUM® HV ) | 0.1 |
| A | PHENOXYETHANOL | 0.6 |
| A | CAPRYLYL GLYCOL | 0.3 |
| B | PEARLS | 40.3 |
| B | LAUROYL LYSIN | 1.7 |
| B | CELLULOSE (CELLULOBEAD®) | 2.4 |
| B | IRON OXIDE (AND) PHYTIC ACID (AND) SODIUM HYDROXIDE (UNIPURE® PHY) | 4.4 |
| B | TITANIUM DIOXIDE (AND) SILICA (SPHERICA HC WHITE 30) | 6.3 |
| C | UNDECANE and TRIDECANE (CETIOL® ULTIMATE MB) | 2.1 |

Phase A: aqueous phase
Phase B: particulate phase (powders)
Phase C: oily phase

To prepare the foundation presented in the table above, the ingredients of Phase A comprising an excess of water are mixed together, which are cold dispersed with a Rayneri at 500 revolutions/minute for 5-10 minutes.

The mixture of powders from phase B is added to phase A, then the whole is dispersed with a Rayneri, gradually increasing the speed to a maximum of 1200 revolutions/minute.

Phase C is added last while continuing to homogenize the paste.

At this stage, the water is present in excess in the slurry, in a quantity of 83% by weight compared to the sum of all the other ingredients of the mixture.

The shaping is carried out by filling, in Top Fill mode, silicone molds equipped with a support grid. A fabric is placed, then a step of compression (Pressure = 2 bars) and suction of the solvent (Pressure = 1 bar) is carried out for 20 seconds

The mold is placed in the freezer for 1 hour. After demolding, the composition is placed in an oven at 45° C. for one hour.

After demolding, the composition is placed in a sealed pack.

### Example 8 Matte Powder for Skin Care

A white powder is prepared for skin care. The final formula of the composition is shown in Table 9 below:

TABLE 9

| Phase | Ingredients | % by weight |
|---|---|---|
| A | WATER | 30 |
| A | GLYCEROL | 10.8 |
| A | HECTORITE (BENTONE®) | 0.1 |
| A | MAGNESIUM ALUMINUM SILICATE (VEEGUM® HV ) | 0.1 |
| A | HYDROXYACETOPHENONE | 0.1 |
| A | CAPRYLYL GLYCOL | 0.5 |
| A | CHLORPHENESIN | 0.3 |
| A | 1,2 HEXANEDIOL | 1.0 |
| B | MICA (SERICIT® M) | 35.1 |
| B | LAUROYL LYSIN | 7.0 |
| B | CELLULOSE (CELLULOBEAD®) | 2.4 |
| B | ETHYLHEXYLGLYCERIN (SENSIVA® SC 50) | 0.5 |
| B | SILICA (AND) TITANIUM DIOXIDE ( SPHERICA® HC WHITE) | 8.6 |
| C | UNDECANE and TRIDECANE (CETIOL® ULTIMATE MB) | 3.5 |

To prepare the compact powder, the ingredients of Phase A are mixed together, which are cold dispersed in a Rayneri at 500 revolutions/minute for 5-10 minutes.

The mixture of powders from phase B is added to phase A comprising an excess of water, to the previous phase, then the whole is dispersed with a Rayneri, gradually increasing the speed up to a maximum of 1200 revolutions/minute.

Phase C is added last while continuing to homogenize the paste.

At this stage, the water is present in excess in the slurry, in a quantity of 86% by weight compared to the sum of all the other ingredients of the mixture.

The shaping is carried out by filling, in Top Fill mode, silicone molds equipped with a support grid. A fabric is placed on the slurry on the surface of the mold, then a step of compression (Pressure = 2 bars) and simultaneous suction of the solvent (Pressure = 1 bar) is carried out for 20 seconds.

The mold is placed in the freezer for 1 hour.

After demolding, the composition is placed in an oven at 45° C. for one hour.

The composition is finally placed in a pack of the “cushion” type.

Once applied to the skin, the composition produces a surprising matte finish related to the presence of SERICITE M, in addition to a hydrating effect for the skin.

1. A cosmetic composition for caring for and/or making up keratin materials in the form of a solid composition comprising, in a physiologically acceptable medium, at least:
water in an amount of from 5% to 50% by weight relative to the total weight of the composition,
at least one hydroxyl compound selected from polyols, glycol ethers and mixtures thereof,
a particulate solid phase comprising at least fillers,
and optionally an oily phase comprising at least one oil,
said composition being obtained by a method comprising the following steps: (i) mixing the particulate phase, and the hydroxyl compound, the water and the optional oily phase constituting the liquid phase, to form a paste; and (ii) shaping the paste by compacting and partial removal of the liquid phase to give the composition in the form of a solid composition.

2. The cosmetic composition according to claim 1, wherein it comprises water in an amount of from 10% to 50%, by weight relative to the total weight of the composition.

3. The composition according to claim 1, wherein it comprises, in a total amount of polyol(s) and/or glycol ether(s) of from 5% to 20%, by weight relative to the total weight of the composition.

4. The composition according to claim 1, wherein the particulate phase comprises fillers selected from platelet fillers, spherical fillers and mixtures thereof.

5. The composition according to claim 1, further comprising hydrophilic treated pigments.

6. The cosmetic composition according to claim 1, further comprising suspending agents, and optionally cohesion agents .

7. The composition according to claim 1, wherein the composition is in the form of a solid hydrating care composition or a solid hydrating make-up composition for the face and/or the lips .

8. The composition according to claim 1, wherein the composition has 3D relief patterns on the surface.

9. A method for manufacturing a solid composition as defined in claim 1, comprising the following steps: (i) mixing the particulate phase, and the hydroxyl compound, the water and the optional oily phase constituting the liquid phase, to form a paste; and (ii) shaping the paste, by compacting and partial removal of the liquid phase present in the paste.

10. A cosmetic method for caring for and/or making up keratin materials, more particularly the skin, comprising the application to the keratin materials of a composition as defined according to claim 1.

11. The cosmetic composition according to claim 6, wherein the suspending agents are selected from the group consisting of hectorite, bentonite and mixtures thereof, and the cohesion agents are selected from the group consisting of magnesium stearate, zinc stearate, magnesium myristate and mixtures thereof.

12. The composition according to claim 7, wherein the composition is in the form of a blush, an eye shadow, a highlighter, a bronzing powder, a protective foundation powder, a concealer, a lip powder or an eyebrow powder.
